(19) United States
(12) Patent Application Publication
(10) Pub. No.: US 2005/0287208 A1
(43) Pub. Date: Dec. 29, 2005

(54) Cross-linked powered/microfibrillated cellulose II

(75) Inventors: Vijay Kumar, Coralville, IA (US); Maria De La Reus Medina, Iowa City, IA (US); Hans Leuenberger, Pffiugeu (CH)

Correspondence Address: MCKEE, VOORHEES & SEASE, P.L.C., 801 GRAND AVENUE, SUITE 3200, DES MOINES, IA, 50309-2721, US

(73) Assignee: University of Iowa Research Foundation, Iowa City, IA (US)

(21) Appl. No.: 11/159,527

(22) Filed: Jun. 22, 2005

Related U.S. Application Data
(60) Provisional application No. 60/582,041, filed on Jun. 22, 2004.

(51) Int. Cl. A61K 9/20; C08B 5/00

(52) U.S. Cl. 424/464; 536/33

(57) ABSTRACT

A new cellulose excipient, UICEL-XL, suitable for use as a binder, filler, and/or disintegrant in the development of solid dosage forms is described. UICEL-XL incorporates a cross-linking agent which provides the excipient with a high degree of crystallinity, high water affinity, and a high specific surface area, thus providing good disintegration properties. UICEL-XL, however, also has the unique distinction of being an effective binder due to its high tensile strength.

### PRIORITY CLAIM

This application claims priority to U.S. patent application Ser. No. 60/582,041 filed Jun. 22, 2004, the disclosure of which is hereby specifically incorporated by reference.

### FIELD OF THE INVENTION

This invention relates to cross-linked powdered/microfibrillated cellulose II, methods of its manufacture, and its uses as an excipient.

### BACKGROUND OF THE INVENTION

Tablets are widely used because they are convenient, easy to use, portable, and less expensive than other oral dosage forms. The ideal tabletting excipient should possess all of the following characteristics: excellent compressibility, adequate powder flow, good disintegration, physiologically safe, inert, and acceptable to regulatory agencies, physically and chemically stable, compatible with other excipients and active excipients, high diluent potential, and inexpensive. Currently, there is no single excipient that fulfills all of these optimum tabletting requirements. Therefore, the search for multifunctional tabletting excipients represents a challenging research area.

Cellulose, the most abundant natural polymer, is a linear homopolymer consisting of 1,4-linked β-D-glucose repeat units. It is widely used as a raw material to prepare a number of excipients. There are four polymorphs of cellulose, namely cellulose I, II, III and IV. Of these, cellulose I is the most prevalent. Cellulose II is typically prepared by mercerization and is the most stable allomorph of cellulose.

Microcrystalline cellulose (MCC), cellulose I powder, is perhaps the best filler-binder currently available. It was first introduced in 1964 under the brand name Avicel® PH and marketed by the FMC Corporation (Philadelphia, Pa.). Since 1992, Avicel® PH has been available in seven grades (Avicel® PH-101, Avicel® PH-102, Avicel® PH-105, Avicel® PH-112, Avicel® PH-113, Avicel® PH-301, and Avicel® PH-302). These grades differ in particle size and moisture content. Currently, MCC is available from different vendors under different trade names.

MCC is prepared by hydrolysis of native (α-cellulose, a fibrous, semicrystalline material, with dilute mineral acids. During hydrolysis, the accessible amorphous regions are removed and a level-off degree of polymerization product is obtained. MCC serves as an excellent binder and possess high dilution potential. However, it suffers from high sensitivity to moisture and lubricants. Addition of a lubricant in the formulation is required especially when a high speed tablet machine is used. MCC also shows poor flow and inconsistent disintegration properties.

Because of the strong hydrogen bonds that occur between cellulose chains, cellulose does not melt or dissolve in common solvents. Thus, it is difficult to convert the short fibers from wood pulp into the continuous filaments needed for artificial silk, an early goal of cellulose chemistry. Today, the cross-linking of cellulose is a crucial textile chemical process, and provides the textile manufacturer a multitude of commercially important textile products. The most commonly used cross-linking systems are based on N-methylol chemistry. Cross-linked cellulose is also used in the pharmaceutical industry.

MCC occurs as a white odorless, tasteless crystalline powder composed of porous particles of an agglomerated product. Apart from its use in direct compression, microcrystalline cellulose is used as a diluent in tablets prepared by wet granulation, as a filler in capsules and for the production of spheres. In the pharmaceutical market, microcrystalline cellulose is available under the brand names Avicel™, Emcocel™, MCC SANAQ®, Ceolus® KG and Vivacel™.

Internally cross-linked form of sodium carboxymethylcellulose (available under the brand name Ac-Di-Sol™) is used as a pharmaceutical disintegrant in both direct compression and wet granulation formulations. Also known as croscarmellose sodium, Ac-Di-Sol™ differs from soluble sodium carboxymethylcellulose only in that it has been cross-linked to ensure that the product is essentially water-insoluble. It is an odorless, relatively free-flowing, white powder.

UICEL™ is a relatively new cellulose-based tabletting excipient, developed by treating cellulose powder with an aqueous solution of sodium hydroxide and subsequent precipitation with ethyl alcohol [Kumar, V., Reus-Medina, M., Yang, D., Preparation, characterization, and tableting properties of a new cellulose-based pharmaceutical aid. Int. J. Pharm. 2002, 235, 129-140; M. Reus, M. Lenz, V. Kumar, and H. Leuenberger, Comparative Evaluation of Mechanical Properties of UICEL and Commercial Microcrystalline and Powdered Celluloses, J. Pharm. Pharmacol., 56, 951-958 (2004); V. Kumar, Powdered/Microfibrillated Cellulose, U.S. Pat. No. 6,821,531]. UICEL is similar in structure to MCC and powdered celluloses (PC). It, however, shows the cellulose II lattice, while MCC and PC belong to the cellulose I polymorphic form. UICEL consists of a mixture of aggregated and/or non-aggregated fibers, depending on the cellulose source used in its manufacture. Compressed tablets formulated with UICEL have the distinction of disintegrating within 15 seconds irrespective of the compression pressure used. Tablets formulated with UICEL have superior disintegration properties. In this regard, tablets prepared using this material, irrespective of the compression pressure employed to prepare them, disintegrate rapidly (in less than 30 seconds) in water. However, this material displays a lower compactability than commercial cellulose I powders.

It is a primary objective of the present invention to provide novel, cross-linked cellulose II with superior disintegration and binding properties.

It is a further objective of the present invention to provide novel, cross-linked cellulose II with potential application as a pharmaceutical excipient.

It is a further objective of the present invention to provide novel, cross-linked cellulose II, and novel dosage forms of the same.

It is yet a further objective of the present invention to provide novel, cross-linked cellulose II having a high crushing strength and a short disintegration time.

The method and means of accomplishing each of the above objectives as well as others will become apparent from the detailed description of the invention which follows hereafter.

### SUMMARY OF THE INVENTION

The present invention relates to the use of cross-linked powdered/microfibrillated cellulose II as a new pharmaceutical excipient. This novel cellulose excipient, UICEL-XL, incorporates glutaraldehyde, polyaldehyde, or polycarboxylic acid as a cross-linking agent. In comparison to UICEL-PH (a cellulose II non-cross-linked powder prepared using Avicel PH-102, the commercial microcrystalline cellulose product, as the starting material according to procedure disclosed in U.S. Pat. No. 6,821,531), UICEL-XL has a high degree of crystallinity, as well as a much higher specific surface area. UICEL-XL is manufactured by combining cellulose II with one or more of the above-referenced cross-linking agents, preferably at high temperature. The cellulose is preferably reacted with the glutaraldehyde in an acidic medium, and for a time period of at least four hours. Like UICEL-PH, UICEL-XL is an effective disintegrant. UICEL-XL, however, also has the unique distinction of being an effective binder due to its high tensile strength.

### BRIEF DESCRIPTION OF THE DRAWINGS

FIG. 1 shows the relationship between Young's modulus and tensile strength values of UICEL-XL and various microcrystalline celluloses (Hydrocellulose, Avicel® PH-102, and Ceolus®), and powdered cellulose (Solka Floc®) and non-cross-linked UICEL (UICEL-HC, UICEL-PH, UICEL-SF, and UICEL-C) products. The non-cross-linked UICEL-HC, UICEL-PH, UICEL-SF, and UICEL-C products were prepared from hydrocellulose, Avicel® PH-102, Solka Floc®, and Ceoluse, respectively.

FIG. 2 illustrates the crushing strength and disintegration time of UICEL-XL tablets made using the cross-linked cellulose II products prepared at 70, 100, and 120° C. in 0.01N HCl. The reaction duration was 6 hours and the weight ratio of cellulose to glutaraldehyde was 1:0.7 (w/w).

FIG. 3 illustrates the effect of different ratios of cellulose and glutaraldehyde in the reaction on the crushing strength and disintegration properties of UICEL-XL. The reaction was carried out at 100° C. for 5 h in the presence of 0.01 N HCl.

FIG. 4 illustrates the effect of reaction time on the crushing strength and disintegration properties of UICEL-XL tablets. The reaction was carried out at 100° C. in 0.01 N HCl using a 1:0.7 weight ratio of cellulose to glutaraldehyde.

FIG. 5 shows the powder X-ray diffractograms of UICEL-PH (A) and UICEL-XL (B).

FIG. 6 shows the FTIR spectra of UICEL-XL and UICEL-PH.

FIG. 7 shows the carbon-13 CP-MAS NMR spectra of UICEL-XL and UICEL-PH.

FIG. 8 shows the sorption/desorption isotherms of UICEL-PH and UICEL-XL. They were obtained using the VTI symmetrical water sorption analyzer.

FIG. 9 shows the “in-die” and “out-of-die” Heckel plots for UICEL-XL. Tablets, which were 11 mm in diameter and weighed about 400 mg each, were prepared using a Carver press at different compression forces and a dwell time of 30 sec.

FIG. 10 shows the elastic recovery profiles for compacts of cellulose excipients.

FIG. 11 shows the disintegration profiles of UICEL-XL and UICEL-PH (UICEL-102). As the compression pressure increased the disintegration time increased.

### DETAILED DESCRIPTION OF THE PREFERRED EMBODIMENT

The present invention relates to the preparation of a cross-linked cellulose II product suitable for use as a direct compression excipient. In a previous patent, U.S. Pat. No. 6,821,531, the disclosure of which is specifically incorporated herein by reference, the inventor describes the synthesis of UICEL-PH, a non-cross-linked cellulose II product.

The use of covalent bonding between the cellulose chains is the most important route to modify the polymer skeleton of cellulose. As noted above, it is widely employed on an industrial scale to improve the performance of cellulose textiles and in the paper industry. Although cellulose is characterized by a self-cross-linking via intermolecular hydrogen bonds, these interactions are reversible in the presence of water. Therefore, covalent cross-linking between cellulose chains avoids undesirable changes of cellulosic structure in the wet state.

There are two methods used to cross-link cellulose: wet- and dry-cross-linking. In wet-cross-linking, the cellulose fibers in the swollen state are treated with the cross-linking agent. In dry-cross-linking, the cellulose fibers are collapsed, i.e., the fibers are collapsed when the water used to swell them is removed, at the time of cross-linking. In the present invention, cellulose II is preferably cross-linked using the wet method.

Cross-linked materials can be lightly or densely cross-linked. Currently, cross-linked sodium carboxymethylcellulose (e.g., Ac-Di-Sol®-FMC BioPolymer, Philadelphia, Pa.) is the only cellulose-based disintegrant commercially available.

UICEL-XL preferably employs a dialdehyde cross-linking agent, with glutaraldehyde being most preferred. Other appropriate cross-linking agents include polyaldehydes, aldehyde-functionalized monosaccharides, disaccharides, and polysaccharides, polycarboxylic acids, etc. The cross-linking agent of this invention should be at least di-functional. Other appropriate cross-linking agents include, but are not limited to, methyolated nitrogen compounds, halohydrins, epoxides, diepoxides, diisocyanates, dihalogen containing compounds, etc.

Cellulose is a weak nucleophile. Glutaraldehyde and/or the other possible cross-linking agents react with cellulose to produce the cross-linked product. Under acidic conditions, aldehyde cross-linking agents are more reactive, facilitating nucleophilic addition of cellulose to the carbonyl group to produce the product, which consists of a mixture of aggregated and non-aggregated fibers. When using non-aldehyde cross-linking agents, it is often advantageous to also employ a coupling agent. Acceptable coupling agents include, but are not limited to, 1,3-dicyclohexylcarbodiimide (DCC), 1-ethyl-3-(3-dimethylaminopropyl)carbodiimide or water soluble carbodiimide, and carbonyldiimidazole (CDI). Additionally, N-hydroxysuccinimide may be added to the reaction mixture to obtain better reaction efficiency. The use of coupling agents is well known and well understood in the art.

In comparing the morphology of UICEL-XL, UICEL-PH and Avicel® PH-102, Avicel® PH-102 has an aggregated structure, composed of small fibers with coalesced boundaries. UICEL-PH has a similar morphology to that of Avicel® PH-102. However, UICEL-PH particles seem to have rougher surfaces than those of Avicel® PH-102. UICEL-XL, in contrast, shows de-aggregated particles (compared to UICEL-PH).

The morphology of UICEL-PH tablet particles looks similar to that of powder particles, i.e., the particles are closely packed, but there appears to be little or no coalescence between boundaries of the particles. In comparison, the cross-sectional view of the Avicel® PH-102 tablet shows coalescence of the particles on the tablet edges. In the center region of the tablet, particles appear deaggregated and show some voids between them. The coalescence between particles results due to the high degree of plasticity of Avicel® PH-102. The cross-sectional view of UICEL-XL tablets illustrate that the edges of the tablet appear similar to that of Avicel® PH-102. However, the central part of the tablet shows more fine, coalesced particles, with very little or no voids between them. This is because UICEL-XL is less ductile than Avicel® PH-102, but more ductile than UICEL-PH.

UICEL-XL has a degree of crystallinity of 75% or greater and, more preferably, 80% crystallinity or greater. It contains the cellulose II lattice.

UICEL-XL has a specific surface area (SSA) of 10 m²/g or greater and, more preferably 15 m²/g or greater and, most preferably 17 m²/g or greater. The specific surface area of UICEL-XL is significantly higher compared to that of UICEL-PH or Avicel® PH-102. This is due to the deaggregation of the particles, as well as a decrease in the degree of polymerization of UICEL-XL during manufacturing. The true densities of the three materials are comparable. The bulk and tap densities of UICEL-XL are lower compared to those of UICEL-PH but higher than that of Avicel® PH-102. UICEL-XL is more porous than UICEL-PH. Avicel® PH-102 has similar porosity as UICEL-XL. The Hausner ratio, Carr index and flow rate results show improved flow of UICEL-XL compared to that of Avicel® PH-102. UICEL-XL shows similar flow as UICEL-PH, suggesting that the cross-linking reaction does not influence the flow rate of UICEL, in general.

All tablets comprising 100% by weight UICEL-XL, irrespective of the reaction temperature used to prepare the product being used, show a disintegration time of less than 100 seconds. However, the disintegration times of all tablets made to the same solid fraction are comparable (less than 20 seconds). In practice, the concentration of UICEL-XL used in the dosage form will depend upon a number of factors, including amount and type of drug incorporated. As a general guideline, the inventors have found that tablets incorporating about 20% by weight UICEL-XL will disintegrate in about 200 seconds.

For tablets formed using UICEL-XL, as a general rule, as the compression pressure increases, the disintegration time also increases. Typically, tablets are made at ˜100 MPa. The disintegration time of UICEL-XL tablets made at this compression pressure is ˜40 sec. In comparison, tablets made using UICEL-PH at the same compression force typically show a disintegration time of 15 sec.

Surprisingly, in addition to its outstanding properties as a disintegrant, and unlike UICEL-PH, UICEL-XL is also an outstanding binder. Tablets that incorporate UICEL-XL have a crushing strength of between about 20-55 kp, preferably 28-55, and most preferably 35-50 kp. In comparison, the non-cross-linked product, UICEL-PH, typically produces tablets with significantly reduced crushing strength values (9-26 kp).

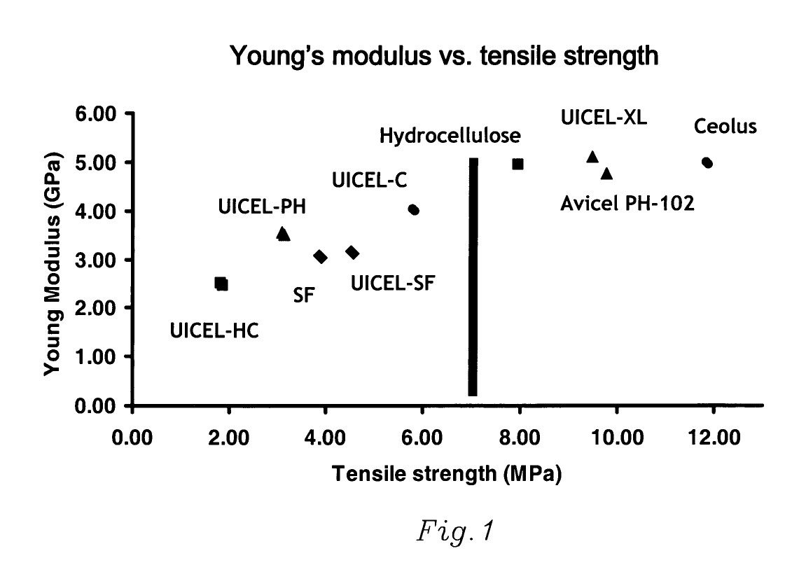

FIG. 1 shows the relationship between Young's modulus and tensile strength values of UICEL-XL and various microcrystalline celluloses (Hydrocellulose, Avicel®, and Ceolus®), powdered cellulose (Solka Floc® (SF)) and non-cross-linked UICEL (UICEL-HC, UICEL-PH, UICEL-SF, and UICEL-C) products. The non-cross-linked UICEL-HC, UICEL-PH, UICEL-SF, and UICEL-C products were prepared from hydrocellulose, Avicel® PH-102, Solka Floc®, and Ceolus®, respectively. Solka Floc® (SF) is a fibrous microcrystalline cellulose product prepared by mechanical disintegration of cotton linter or cellulose pulp. Other materials were produced by hydrolysis of cellulose. The viscosity average degree of polymerization of SF was about 900, while MCC products had a DP value between 150 and 350.

The Young's modulus and tensile strength values of UICEL-XL are much higher than that of UICEL products and Solka Floc®, but comparable to those of hydrocellulose, Avicel®, and Ceolus®. The lower Young's modulus and tensile strength values obtained for Solka Floc® compared to various microcrystalline cellulose products is attributed to its fibrous nature and more brittle character. The lower the Young's modulus, the more elastic the material. UICEL-XL has a lower tendency to recover elastically than UICEL-PH. By cross-linking, the cellulose chains become rigid, and, as a result, their mobility decreased. In general, the stiffer the structure is, the lower the elasticity. This could explain the high Young's modulus value and lower elastic recovery tendency observed for UICEL-XL compared to that of UICEL-PH, which lacks these additional interchain bonds, and hence, displays more flexibility and elasticity. UICEL-XL and Avicel®-PH-102 show comparable elastic recovery.

UICEL-XL forms stronger tablets than UICEL-PH. A comparison of the tensile strengths of UICEL-PH and UICEL-XL tablets shows that the cross-links made the molecule more compactable. This indicates that by modifying the elasticity of cellulose II powders, the binding properties can be altered. In other words, by reducing the elasticity of UICEL via cross-links, more interparticulate bonds survive during decompression, and consequently, increase the tensile strength of the compact, compared to the non-cross-linked UICEL compacts.

The UICEL-XL of this invention is manufactured by combining a source of cellulose with at least one of the cross-linking agents enumerated above, at a temperature ranging from about 60-130° C. Preferably, the cellulose and cross-linking agent(s) are combined at a weight ratio of 1:0.07 and the reaction is conducted at a temperature of about 100° C. for a period of 8.5 hours. Persons skilled in the art would readily understand that the described ratios, temperatures and reaction times can vary greatly depending upon the use and purpose of the composition. Further, varying one factor will allow other factors to be modified. For example, a higher temperature allows shorter reaction time. A lower concentration of cross-linking agent could also be used and still comparable results. As a general rule, the higher the reaction temperature and/or the length of the reaction, the higher the crushing strength of the UICEL-XL.

This cellulose can originate from any source, including cotton linters, alpha cellulose, hard and soft wood pulp, regenerated cellulose, amorphous cellulose, low crystallinity cellulose, powdered cellulose, mercerized cellulose, bacterial cellulose and microcrystalline cellulose. Illustrative methods can be found in the following publications, the disclosures of which are hereby incorporated by reference: Powdered cellulose: U.S. Pat. Nos. 4,269,859, 4,438,263, and 6,800,753; Low crystallinity cellulose: U.S. Pat. No. 4,357,467; U.S. Pat. No. 5,674,507; Wei et al. (1996); Microcrystalline cellulose: U.S. Pat. Nos. 2,978,446, 3,146,168, and 3,141,875, Chem Abstr. 111 (8) 59855w, 111 (8) 59787a, 108 (19) 152420y, 104 (22) 188512m, 104 (24) 209374k; CA 104 (24) 193881c, 99 (24) 196859y, 98 (12) 95486y, 94 (9) 64084d, and 85 (8) 48557u. The preferred source of cellulose for use in this invention is cellulose II. However, cellulose I may be used so long as it is first converted to cellulose II, using the technology described in U.S. Pat. No. 6,821,531.

Prior to treatment in accordance with the methods and solvents of this invention, the cellulose II is preferably treated with a swelling agent for 0.5-56 hours, and preferably for about 12-48 hours, at room temperature. The swelling agent should be used in an amount sufficient to soak the cellulose II. Use of the swelling agent increases the rate of reaction and allows the reaction to occur at a lower temperature. Examples of suitable swelling agents include, but are not limited to phosphoric acid, isopropyl alcohol, aqueous zinc chloride solution, water, amines, etc., with water being preferred. Once the cellulose II has swelled sufficiently, the swelling agent is preferably removed by washing with water to prevent any potential incompatibilities with the cross-linking agent.

The cellulose is preferably combined with the cross-linking agent(s) in ratio ranging between about 1:0.01 to about 1:>1 cellulose to cross-linking agent. There is no limit on the upper range of cross-linking agent that may be used, the only limiting factor being practicality and cost. The preferred ratio is between about 1:0.3 to about 1:1 cellulose to cross-linking agent. In general, the higher the ratio of cross-linking agent to cellulose, the higher the crushing strength, but the longer the disintegration time. So, the binding and/or disintegration properties of the UICEL-XL can be easily modified by altering the ratio of cellulose II to cross-linking agent depending upon its intended use.

The cellulose is allowed to react with the cross-linking agent(s) for a time period of at least 2 hours, with about 4-12 hours being preferred, and at least 8.5 hours being most preferred at the optimized temperature. In a preferred embodiment, the cellulose II is reacted with the cross-linking agent with constant stirring and/or agitation. In general, longer reaction times produce UICEL-XL tablets with higher crushing strengths, but longer disintegration times.

As already noted above, combination of the cellulose with an aldehyde cross-linking agent(s) preferably (but not mandatorily) occurs in an acidic medium. In this regard, the pH of the reaction medium is preferably 2.0 or less, with about 1.0 being most preferred. Hydrochloric acid is a preferred acid for this purpose. The only requirements of the acid are that it be capable of protonating carbonyl oxygen without negatively affecting the cross-linking reaction. Also noted above, at least one coupling agent is preferably also included if a non-aldehyde cross-linking agent is employed.

Once the cross-linking reaction is complete, the cross-linked product is filtered from the reaction mixture by conventional means, i.e. filtration, ultrafiltration, etc. The product is then preferably washed to a neutral pH by conventional means, then with a water-miscible organic solvent, such as alcohols, acetone, tetrahydofuran, and acetonitrile, and finally dried. In a preferred embodiment, the product is dried to a 7% or less moisture by weight.

The resulting UICEL-XL may be used as an excipient in the medical, pharmaceutical, agricultural, and veterinary fields. UICEL-XL may be used in the manufacture of solid dosage forms, such as granules, microspheres, tablets, capsules, etc. As noted above, UICEL-XL has both excellent disintegrant and binding properties.

The formulation of pharmaceutically-acceptable dosage forms is well known in the art. As used herein, the term “pharmaceutically-acceptable” refers to the fact that the preparation is compatible with the other ingredients of the formulation and is safe for administration to humans and animals.

Oral dosage forms encompass tablets, capsules, and granules. Preparations which can be administered rectally include suppositories. Other dosage forms include suitable oral compositions which can be administered buccally or sublingually. The manufacture of such preparations is itself well known in the art. For example pharmaceutical preparations may be made by means of conventional mixing, granulating, and lyophilizing processes. The manufacturing processes selected will depend ultimately on the physical properties of the active ingredient used.

The following examples are provided to illustrate but not limit the invention. Thus, they are presented with the understanding that various modifications may be made and still be within the spirit of the invention.

### EXAMPLE 1

### Preferred Method of Manufacturing UICEL-XL

Starting cellulose II (200 g) was soaked in water (600 mL water). 124 mL of glutaraldehyde was then added to the hydrated cellulose suspension. The mixture was heated to 100° C. at a constant stirring for 8.5 h. The reaction mixture was filtered and the white residue obtained was washed first with water to a neutral pH and then with acetone. The product was dried at 55-60° C. to a moisture content of ≦7%.

### EXAMPLE 2

### Preferred Method of Manufacturing UICEL-XL

Materials and Methods

Materials

UICEL-PH was prepared using Avicel® PH-102 as the starting material. The method of preparation has been discussed in detail in Kumar et al., Preparation, characterization, and tabletting properties of a new cellulose-based pharmaceutical aid. Int. J. Pharm., 2002, 235, 129-140. Glutaraldehyde and concentrated hydrochloric acid were purchased from Fisher Scientific (Fair Lawn, N.J.) and Spectrum Quality Products Inc. (New Brunswick, N.J.), respectively. Avicel® PH-102 was from FMC Corporation (Philadelphia, Pa.).

Preparation of Modified UICEL

50 grams of UICEL-PH powder were put in a three-neck round bottom flask, equipped with a condenser, a stirrer, and a stopper. 300 mL of distilled water was added to the powder and the mixture was allowed to stand at room temperature for a period of 12 hours. To the hydrated UICEL-PH suspension, an appropriate amount of 1 N hydrochloric acid, equivalent to give a final acid concentration of 0.01 N, was added. This was followed by addition of glutaraldehyde solution (50% w/w), equivalent to a weight-by-weight ratio of cellulose to glutaraldehyde of 1:0.3 or 1:0.7. The mixture was heated at 70, 100, or 120° C., with constant stirring, for 4, 6, or 8.5 hours. The reaction mixture cooled to room temperature and then filtered and then washed first with water until the pH of the washing was around 7 and then with acetone (cellulose powder: acetone=1:0.5 w/v). The product was finally collected on a Buckner funnel and air dried at 60° C. in a convection oven (Thelco Model 4, GCA/Precision Scientific) until the moisture content of the powder was <7%.

Degree of Crystallinity

The powder X-ray diffraction (XRD) measurements were conducted over a 5-40° 2θ range on a Siemens Model D5000 diffractometer, equipped with monochromatic CuK α (α₁=1.54060 Å, α₂=1.54438 Å) X-rays. The step width was 0.0200° 2θ/min with a time constant of 0.5 sec. The integration of the crystalline reflections was achieved using the Diffrac^{Plus} diffraction software (Eva, Version 2.0, Siemens Energy and Automation, Inc. Madison, Wis.). The degree of crystallinity of samples was expressed as the percentage ratio of the integrated intensity of the sample to that of crystalline cellulose II standard, which was prepared by triple mercerization of cotton linter followed by treatment with 1 N HCl at boiling temperature for 8 hours. It has been found that repeated rather than prolonged swelling-deswelling is preferred in order to remove the last traces of cellulose I. Since no other synthetic or natural 100% crystalline cellulose II standard is currently commercially available, this material can be used as an acceptable reference in the crystallinity determinations.

Degree of Polymerization

The degree of polymerization of samples was determined by the viscosity method and the procedure has been described by Kumar et al. previously. Kumar et al., Preparation, characterization, and tabletting properties of a new cellulose-based pharmaceutical aid. Int. J. Pharm., 2002, 235, 129-140.

FTIR

The FT-IR spectra of products were obtained as KBr pellets on a Nicolet 5DXB infrared spectrophotometer (Nicolet Instruments Corp., Madison, Wis.).

NMR

The solid-state ¹³C CP/MAS NMR spectra of the samples were obtained on a Bruker MSL-300 spectrometer at room temperature, with a 4 μs pulse for proton polarization, 4 ms contact time for polarization transfer and a 1 s pulse delay. A total of 512 data were collected for frequency induction decay (FID) and a line broadening of 50 Hz was applied to the spectra. The region between 0 and 200 ppm was plotted. There were no peaks above 200 ppm. The number of scans used to obtain the spectra was 4000.

SEM

The SEM photographs of the samples were obtained using a Hitachi S-4000 microscope (Hitachi Ltd., Tokyo, Japan). The samples were loaded on aluminum stubs covered with a double-sided tape. They were then coated with a gold/palladium (60/40) mixture for 4 min in an Emitech K550 coater (Emitech Products, Inc. Houston, Tex.). Photographs were taken and processed.

Specific Surface Area and Densities

Surface area measurements were performed using a Quantasorb Sorption System (Quantachrome Corp., Boynton Beach, Fla.). Helium gas was used as the carrier, and nitrogen gas as the adsorbate. A five point BET analysis was conducted on all samples, by performing the adsorption and desorption at relative pressures ranges between 0.05 and 0.25. Prior to performing the measurements, all samples were dried at 60° C. under reduced pressure for 24 h prior to analysis. In addition, they were degassed for 12 h at 60° C. under a continuous flow of nitrogen. The pore volume determination was conducted as described above but using a relative pressure of 0.97.

The true, bulk and tapped densities were determined as described in Kumar et al. (2002).

Water Vapor Sorption Studies

The equilibrium moisture curves were measured with a Symmetrical Vapor Sorption Analyzer SGA-100 (VTI Corporation, Hialeah, Fla.). Prior to performing the measurements, all samples were dried at 60° C. under reduced pressure for 24 h prior to analysis.

Preparation of Tablets

Tablets of the studied materials, each weighing about 400 mg, were prepared on a Carver hydraulic press at 105 MPa using an 11-mm diameter die and flat-face punches and a dwell time of 30 s. For the Heckel analysis, the pressure range employed was from 15 to 210 MPa.

Heckel Analysis, Young's Modulus, Elastic Recovery and Tensile Strength

The tensile strength of the compacts was determined using the Qtest I™ (MTS, Cary, N.C.) universal tester and the crosshead speed (i.e. rate of load application) of 0.03 mm/s, according to the method developed by Ramsey. Ramsey, P. J. Physical evaluation of the compressed powder systems: the effect of particle size and porosity variation on Hiestand compaction indices. Ph.D. Thesis, The University of Iowa, Iowa City, 1996. The peak load required to cause diametrical splitting of the tablet was then used to calculate the tensile strength according to the equation: σ₀=2P/πDt, where σ₀ is the maximum radial tensile strength, P is the applied load, D is the diameter of the compact, and t is the compact thickness. The elastic modulus, or Young's modulus, E, was determined according to the Hooke's law E=σ/ε, where (y is the axial stress and E is the axial strain. Crushing strengths were determined using a Dr. Schleuniger® Pharmatron tablet hardness tester (Schleuniger Model 8, Manchester, N.H.). The Heckel analysis was conducted in accordance with Reus-Medina et al., Comparative evaluation of the powder properties and compression behavior of a new cellulose-based direct compression excipient and Avicel PH-102. J. Pharm. Pharmacol., 2004, 56, (8), 951-956.

The elastic recovery (ER) of the tablet was determined using the equation: ER=[(Hₜ−H₀)/H₀], where, Hₜ is the height of the tablet 48 hours after compression and H₀ is the height of the tablet in the die at different compression pressures applied. Armstron, N. A. et al., Elastic recovery and surface area changes in compacted powder systems. Powder Technol., 1973, 9, 298-290.

Disintegration Profile

The disintegration test was performed according to the US Pharmacopoeia/National Formulary disintegration method in water at 37° C. using an Erweka GmbH apparatus (type 712, Erweka, Offenbach, Germany). USP, USP 28/NF 23 (United States Pharmaceopeia 28/National Formulary 23). <701> Disintegration, p. 2411, Washington, D.C., 2005.

Results and Discussion

Preparation of UICEL-XL

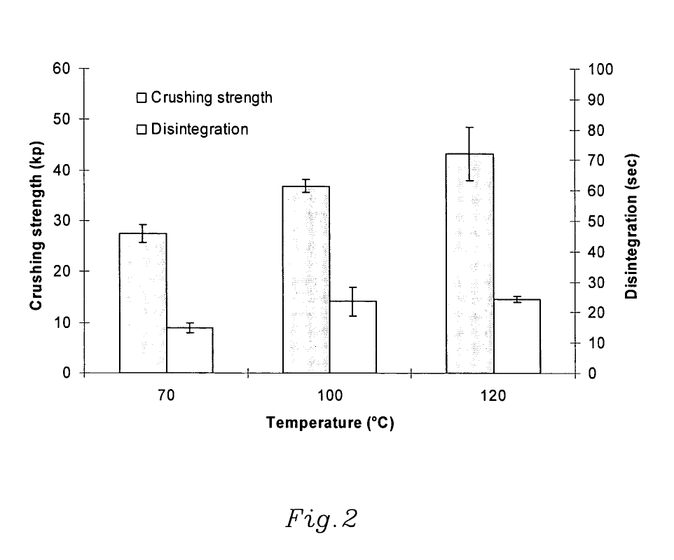

FIG. 2 shows the effect of reaction temperature on the crushing strength and disintegration time of the tablets of the reaction product. These two properties were used as indicators of the success of the reaction since the goal was to improve the binding properties of UICEL-PH while preserving its good disintegration characteristic. UICEL-PH tablets made using a compression force of 4000 lbs had a crushing strength of 21-27 kp, and a disintegration time of less than 15 seconds. The ratio of UICEL-PH:glutaraldehyde used for this study was 1:0.6 (w/v) and the reaction time was 6 hours. An increase in the reaction temperature from 70° C. to 100° C. produced an increase of about 10 kp in the crushing strength of the tablets and an increase in the disintegration time of about 5 seconds. A further increase in the temperature from 100° C. to 120° C. caused a further increase of about 6 kp in the crushing strength and no change in the disintegration time. These results indicate that the higher the reaction temperature the better the binding properties of the product formed. There was no adverse effect on the disintegrant property of the products; irrespective of the temperature used in their manufacture, all compacts disintegrated within 15 seconds, the same disintegration time as was observed for UICEL-PH tablets.

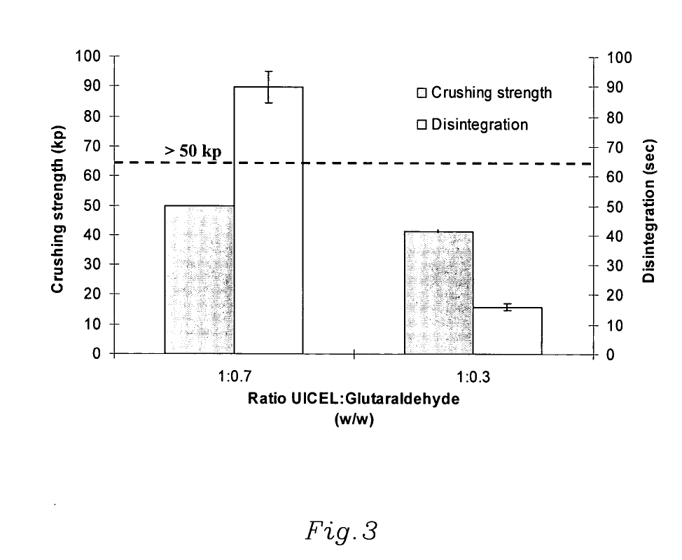

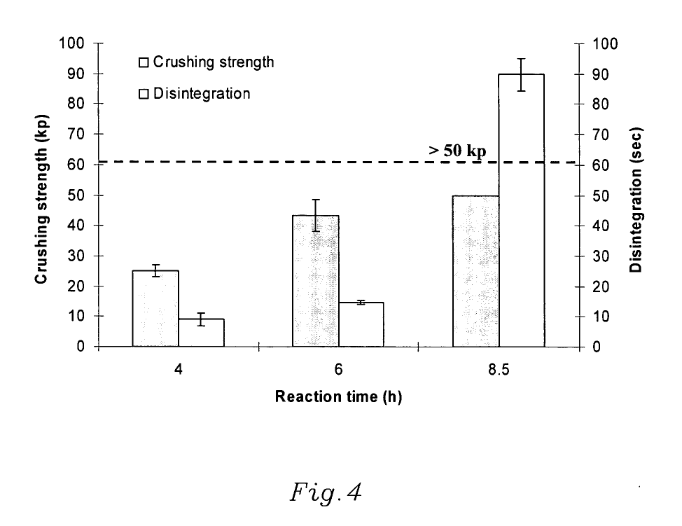

FIG. 3 displays the results of the reactions conducted at different ratios of UICEL-PH and glutaraldehyde (w/v) at 100° C. for 5 hours. As can be seen, a ratio of 1:0.7 of UICEL:glutaraldehyde gave a product, whose tablets showed a crushing strength of greater than 50 kp and a disintegration time of about 90 seconds. FIG. 4 presents the results of the reactions carried out for different periods at 120° C. using a UICEL-PH:glutaraldehyde ratio of 1:0.7 (w/v). An increase of reaction time from 4 hours to 6 hours brought about an increase in the crushing strength of around 20 kp, while the disintegration time remained under 20 seconds. An additional increase in the reaction time from 6 hours to 8.5 hours caused the crushing strength of the compact to increase higher than 50 kp and a disintegration time of about 90 seconds.

Taken together, the above results indicate the following optimized reaction conditions to cross-link cellulose II with glutaraldehyde: a temperature of 100° C., a UICEL-PH:glutaraldehyde ratio of 1:0.7 (w/v) and a reaction time of 8.5 hours.

Characterization of UICEL-XL

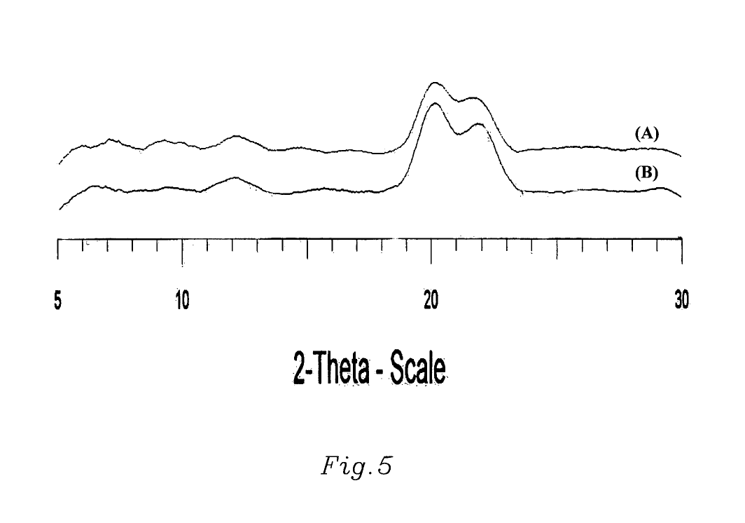

FIG. 5 compares the powder X-ray diffractograms of UICEL-PH (A) and UICEL-XL (B). The presence of a similar peak pattern for UICEL-XL as that of UICEL-PH indicates that UICEL-XL also possesses the cellulose II lattice.

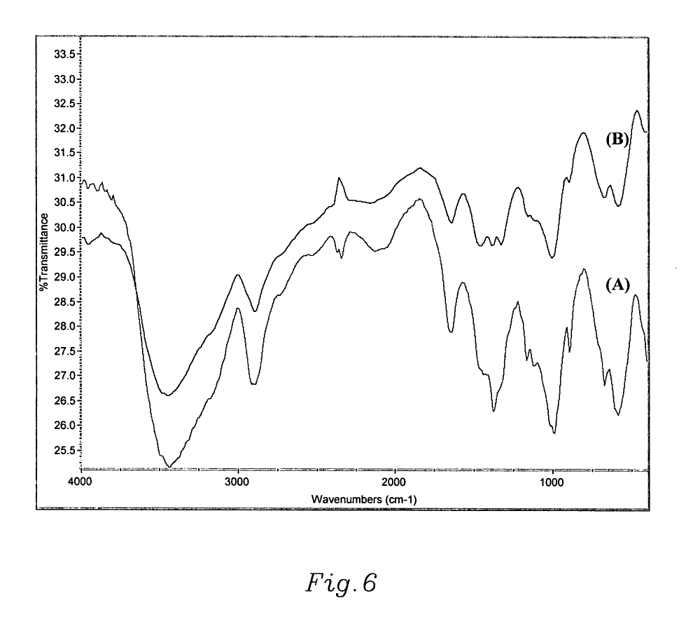

The FT-IR spectra of UICEL-PH and UICEL-XL are compared in FIG. 6. The two spectra appear similar except for the following notable differences: (i) the characteristic intermolecular and intramolecular O—H stretching vibration band in the spectrum of UICEL-XL is slightly less broad, showing the maximum intensity at 3444 cm⁻¹. The corresponding band in the spectrum of UICEL-PH appears at 3427 cm⁻¹. This suggests that some of the OH groups in UICEL-PH have been consumed in cross-linking; (ii) the COO′ stretching band region (1350-1450 cm⁻¹) is less strong for UICEL-XL than for UICEL-PH; and (iii) the absorption band at 892 cm⁻¹ in the spectrum of UICEL-XL is relatively weaker in intensity than that for UICEL-PH. The lower intensity of this band indicates that UICEL-XL has a higher crystallinity and contains the cellulose II lattice. These results are in good agreement with those obtained by the powder X-ray diffraction method. Overall, the reduced intensities of peaks in the region between 590 cm⁻¹ and 1640 cm⁻¹ in the spectrum of UICEL-XL, compared to the corresponding peaks in the spectrum of UICEL-PH, suggest that the hydroxyl groups on the glucose ring have been substituted with the cross-linking agent.

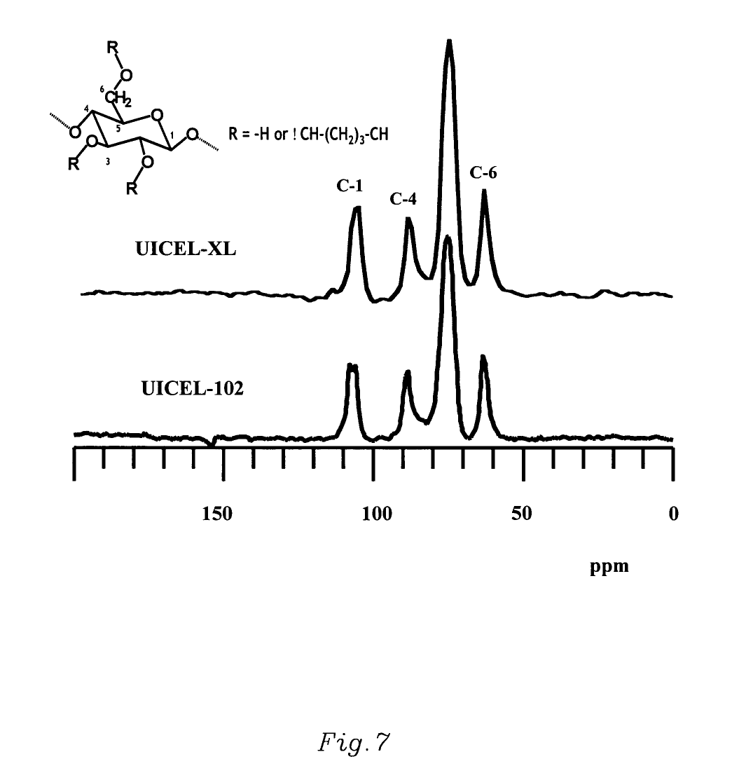

The carbon-13 CP/MAS spectra of UICEL-XL and UICEL-PH are depicted in FIG. 7. The peaks at 101, 89, and 65 ppm in the spectra are due to C1, C4, and C6, respectively. C2, C3, and C5 appear at about 74 ppm. These peaks were assigned on the basis of the spectral data reported in the literature for various unmodified celluloses. The C1 resonance for both materials shows a distinctive pattern; for UICEL-PH the peak splits into two equivalent lines, whereas for UICEL-XL no splitting was observed. The splitting of this peak indicates the presence of two magnetically non-equivalent C1s. A small shoulder at about 115 ppm in the spectrum of UICEL-XL could be due to the glutaraldehyde carbon atom linked to the oxygen atoms. The methylene carbon peaks belonging to glutaraldehyde were expected to be in the range between 20 and 35 ppm. Thus, the small peak appearing at ˜23 ppm in the spectrum of UICEL-XL could be due to these carbons. The small intensity of this peak indicates that UICEL-XL is a lightly cross-linked material.

The degree of crystallinity of the samples was expressed as the percentage ratio of the integrated intensity of the sample to that of a crystalline standard of cellulose II. Table 1 presents the crystallinity values and the degree of polymerization values obtained for UICEL-XL and UICEL-PH. UICEL-XL is more crystalline (˜82%) than UICEL-PH (˜68%). The higher degree of crystallinity of UICEL-XL is not surprising because the cross-linking reaction was done in an acidic medium at a temperature of about 100° C., which hydrolyzed the amorphous portions of UICEL-PH and produced the highly crystalline material. Klemm et al. also reported that cross-linking in an acidic medium at high temperatures brings about some chain degradation due to the hydrolysis of the glycosidic linkages. Klemm, D. et al., Comprehensive cellulose chemistry: Vol. 2: Functionalization of cellulose. Wiley-VCH: New York, 1998; 33-51. The significantly lower DP value of UICEL-XL, compared to that of UICEL-PH, confirms this (Table 1).

TABLE 1
Degree of crystallinity and degree of polymerization
values of cellulose excipients

| | % Crystallinity | DP |
|---|---|---|
| Material | n = 3 (S.D.) | n = 2 |
| UICEL-PH | 68.2 (3.5) | 187 |
| UICEL-XL | 82.5 (2.2) | 79 |

^{a}Mean of two samples from three determinations

The true, bulk and tapped densities of UICEL-XL and UICEL-PH are compared in Table 2. The true densities of both samples are comparable. UICEL-XL, compared to UICEL-PH, had lower bulk and tapped densities and a higher porosity. UICEL-XL consisted of partially deaggregated particles. This occurred due to the acidic reaction medium, high reaction temperature, and vigorous agitation used during the manufacture of the material. According to the results shown in Table 2, UICEL-XL is more porous than UICEL-PH. Thus, the reduced bulk and tapped densities and the higher porosity of UICEL-XL, compared to the corresponding values of UICEL-PH, could be due to different sizes and shapes of deaggregated particles formed as a result of the manufacturing conditions.

TABLE 2
Densities and porosities of cellulose excipients.

| | Density [g/mL] | | | Total |
|---|---|---|---|---|
| | True, n = 3 | Bulk, n = 3 | Tap, n = 3 | porosity |
| Material | (S.D.) | (S.D.) | (S.D.) | [%] |
| UICEL-PH | 1.531 | 0.449 | 0.578 | 62.6 |
| | (0.002) | (0.006) | (0.002) | |
| UICEL-XL | 1.528 | 0.383 | 0.458 | 70.0 |
| | (0.001) | (0.002) | (0.003) | |

The surface area, densities, porosity, and flow properties of UICEL-PH, UICEL-XL, and Avicel PH-102 are shown in Table 3. The BET N₂ surface area and the pore volume of UICEL-XL are significantly higher, about forty times than those of UICEL-PH. This is attributed to its deaggregated structure and decreased degree of polymerization, resulting in smaller particles. Although the pore volume is much higher in UICEL-XL, the difference in the average pore diameters of both materials is not as large.

TABLE 3
Degree of polymerization (DP), Specific Surface Area
(SSA), Densities (ρ), Porosity, and Flow Properties
of UICEL-PH, UICEL-XL, and Avicel PH-102

| | UICEL-XL | | UICEL-PH | | Avicel ® PH-102 | |
|---|---|---|---|---|---|---|
| SSA (m²/g) | 17.8248 | | 0.4774 | | 1.4508 | |
| ρₜᵣᵤₑ (g/mL) | 1.528 | (0.001)^{#} | 1.531 | (0.002)^{#} | 1.577 | (0.004) |
| ρ_{bulk} (g/mL) | 0.383 | (0.002)^{#} | 0.449 | (0.001)^{#} | 0.332 | (0.009) |
| ρₜₐₚ (g/mL) | 0.458 | (0.003)^{#} | 0.573 | (0.003)^{#} | 0.403 | (0.003) |
| Porosity (%) | 70.0 | | 62.6 | | 74.0 | |
| DP | 79* | | 187* | | 201 | |
| Hausner ratio | 1.19 | (0.01) | 1.18 | (0.02) | 1.27 | (0.03) |
| Carr's Index | 16.36 | (0.52) | 15.29 | (1.51) | 21.37 | (2.38) |
| Angle of | 37 | (1) | 36 | (1) | 41 | (2) |
| repose (°) | | | | | | |

| | | | |
|---|---|---|---|
| Flow rate | 12.5^{a} | 13.8^{a} | —^{b} |
| (g/sec) | | | |

^{a}orifice diameter was 11/16″.
^{b}did not pass through the 11.16″ orifice.
*also listed in Table 1;
^{#}also given in Table 2

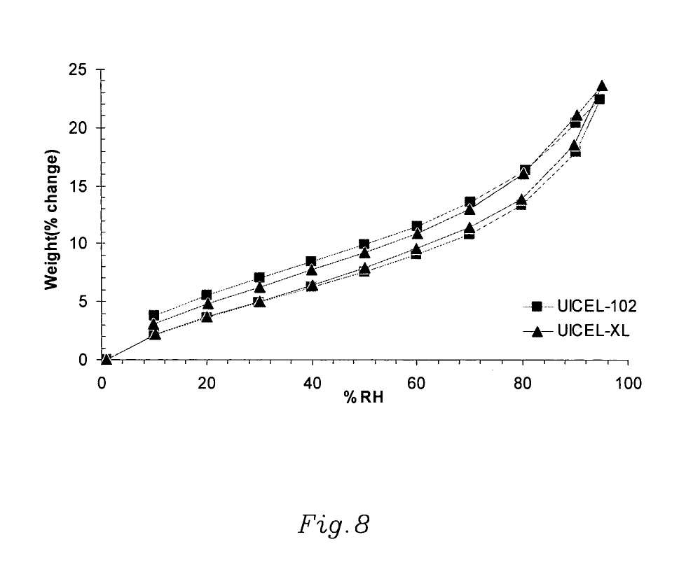

FIG. 8 shows the water sorption isotherms for UICEL-XL and UICEL-PH. Both materials showed comparable water uptake. Table 4 displays the degree of crystallinity and the number of moles of water vapor experimentally observed per gram of UICEL-XL and UICEL-PH. Interestingly, UICEL-XL has a higher crystallinity, but shows comparable affinity towards water; the number of moles of sorbed water experimentally observed was nearly the same as obtained for UICEL-PH. The slightly narrower hysteresis observed for UICEL-XL compared to that for UICEL-PH suggests that water vapor in UICEL-PH is slightly more tightly held. This could be due to the lower degree of crystallinity of UICEL-PH, where sorbed water is surrounded by the crystalline region, which acts as a barrier for the entrapped moisture. It appears that the introduction of glutaraldehyde as a cross-linking agent only slightly changes the crystalline lattice.

TABLE 4
Moles of water vapor sorbed by various celluloses.

| | Degree of | Moles of water |
|---|---|---|
| | crystallinity (%) | vapor/g of cellulose |
| UICEL-PH | 62.4-68.2 | 0.0027 |
| UICEL-XL | 82.5 | 0.0027 |

UICEL-XL and UICEL-PH show comparable accessibility to water despite having different degrees of crystallinity. Interestingly, UICEL-XL and Avicel® PH-102 have comparable degrees of crystallinity, but UICEL-XL is more accessible for water vapor than Avicel® PH-102. It could be that the presence of cross-links in the chains serves as dislocation sites, facilitating penetration of water vapors to sites located within the crystal lattice.

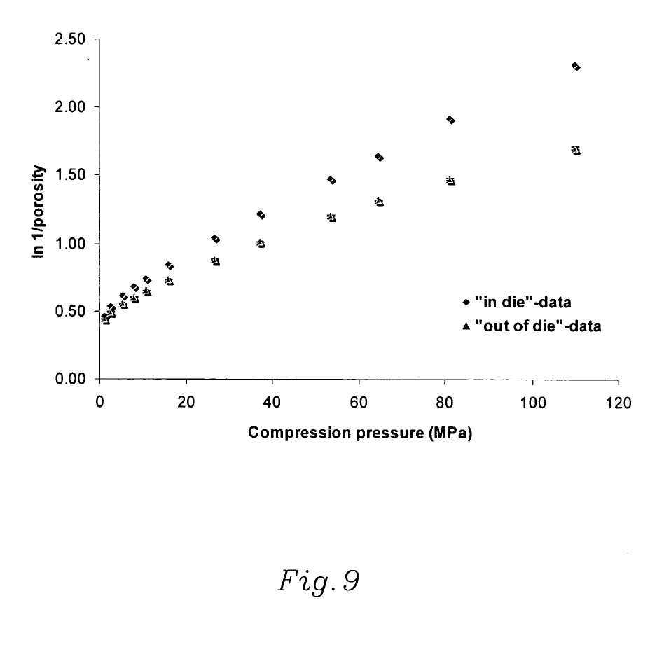

The “in-die” and “out-of-die” Heckel plots for UICEL-XL are shown in FIG. 9. As can be seen from this Figure, the Heckel curves showed a curvature spanning the compression pressure range between 1 MPa and 8 MPa. This was due to the fragmentation and rearrangement of the powder bed. The Heckel parameters for UICEL-XL and UICEL-PH calculated from the “in-die” and “out-of-die” data over the whole compression pressure range employed and from the linear portion of the curves are listed in Table 5. The linear regression analyses of the UICEL-XL “in-die” and “out-of-die” Heckel curves over the whole compression pressure range gave correlation coefficient of 0.994 and 0.982, respectively, corresponding to the mean yield pressures of 59.56 and 85.62 MPa, respectively. Considering the linear region of the curves only, the respective mean yield pressure values for UICEL-XL were 62.50 and 91.74 MPa. The lower “in-die” yield pressure values are due to the elastic deformation contribution. These results show that UICEL-XL is more ductile than UICEL-PH.

TABLE 5
Values of “in-die” and “out-of-die” Heckel equation parameters^{b}

| | Compression | “In-die” | | | | “Out-of-die” | | | |
|---|---|---|---|---|---|---|---|---|---|
| | Pressure | K | | | | K | | | |
| | Range | [10⁻³ | σ_{y}, | | | [10⁻³ | σ_{y}, | | |
| Material | [MPa]^{a} | MPa⁻¹] | [MPa] | A | r² | MPa⁻¹] | [MPa] | A | r² |
| UICEL- | 1-111 | 16.79 | 59.56 | 0.541 | 0.994 | 11.68 | 85.62 | 0.517 | 0.982 |
| XL | | (0.02) | (0.09) | (0.000) | | (0.01) | (0.09) | (0.000) | |
| | 26-81 | 16.0 | 62.50 | 0.614 | 1.000 | 10.9 | 91.74 | 0.605 | 0.996 |
| UICEL- | 1-111 | 13.34 | 74.96 | 0.490 | 0.996 | 7.96 | 125.63 | 0.463 | 0.984 |
| PH^{c} | | (0.27) | (0.02) | (0.013) | | (0.32) | (0.04) | (0.016) | |
| | 37-108 | 12.51 | 79.95 | 0.551 | 1.000 | 6.62 | 151.13 | 0.564 | 0.999 |

^{a}Used in the regression analysis.
^{b}Standard errors of the mean are given in parentheses.
^{c}Taken from Reus-Medina et al.

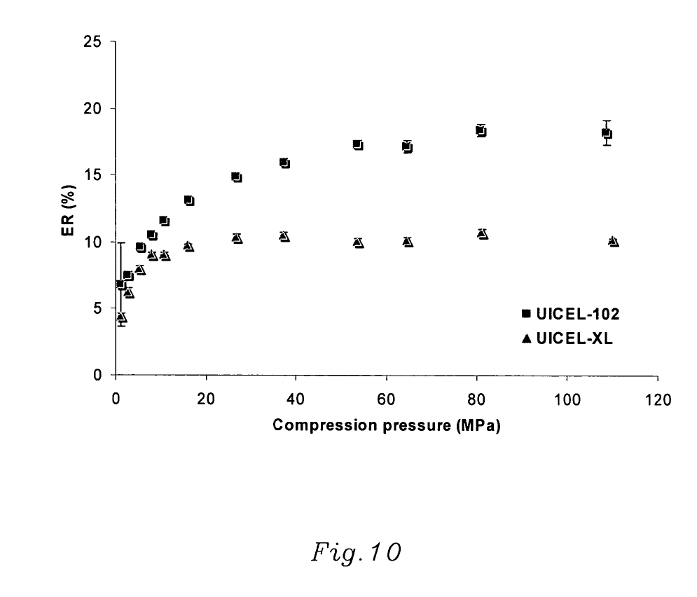

The Young's modulus (E) values are given in Table 6. The lower the Young's modulus, the more elastic the material is. FIG. 10 presents the elastic recovery profiles of UICEL-XL and UICEL-PH (UICEL-102) over the whole compression pressure range used in the study. These results clearly show that UICEL-PH has a greater tendency to recover elastically than UICEL-XL. By cross-linking, the cellulose chains become rigid, and, as a result, their mobility decreased. In general, the stiffer the structure, the lower the elasticity is. This could explain the high Young's modulus value and lower elastic recovery tendency observed for UICEL-XL compared to that of UICEL-PH, which lacks these additional interchain bonds, and hence, displays more flexibility and elasticity.

TABLE 6
Mechanical properties of cellulose excipients

| | Young's modulus | Tensile strength |
|---|---|---|
| | [GPa] | [MPa] |
| | E | σₜ |
| UICEL-XL | 5.57 | 9.48 |
| | (0.63) | (0.16) |
| UICEL-PH | 3.56 | 3.08 |
| | (0.11) | (0.16) |
| Avicel ® PH-102 | 4.76 | 9.77 |
| | (0.15) | (0.31) |

n = 3.
Standard deviations are given in parentheses.

A comparison of the tensile strengths of UICEL-PH and UICEL-XL tablets shows that the cross-links made the molecule more compactable. (See Table 6).

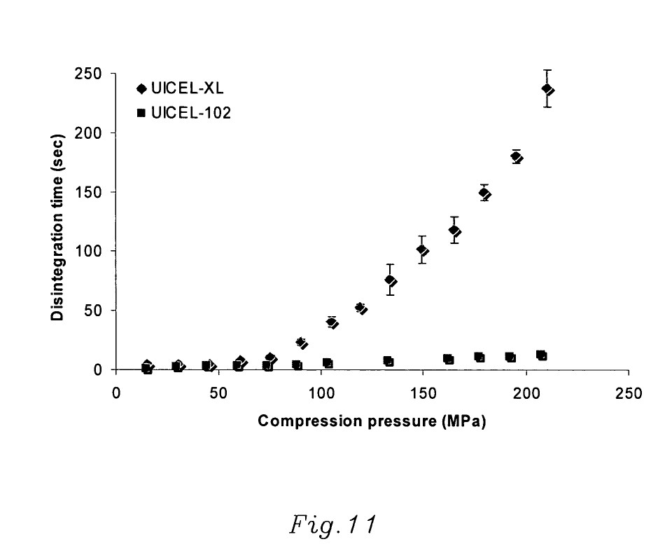

The disintegration profiles of UICEL-XL and UICEL-PH compacts are shown in FIG. 11. As the compression pressure increased, the disintegration time increased for both materials. At the maximum pressure (210 MPa), the disintegration time was about 4 minutes for the UICEL-XL compacts and about 12 seconds for the UICEL-PH compacts. UICEL-XL tablets made at pressures <100 MPa disintegrated in ˜40 seconds. The increase in the disintegration time with an increase in the applied pressure for UICEL-XL is predictable because of the higher tensile strength of its compacts compared to those of UICEL-PH. Table 7 lists the disintegration times of UICEL-XL and UICEL-PH tablets of comparable strengths. The disintegration time of UICEL-XL compacts was about 7 seconds faster than those of UICEL-PH.

TABLE 7
Disintegration values for compacts of cellulose
excipients of comparable strengths

| | Disintegration time^{a} | Crushing strength^{a} |
|---|---|---|
| Material | [sec] | [kp] |
| UICEL-XL | 4.6 | 25.4 |
| | (0.5) | (0.9) |
| UICEL-PH^{c} | 12.7 | 22.0 |
| | (3.4) | (1.0) |

^{a}n = 20.
Standard deviations are given in parentheses

The water vapor sorption results, along with the disintegration results, suggest that the fast disintegration properties are specific to the cellulose II form, rendering the hydroxyl groups to be more accessible for interaction with water molecules. In the case of UICEL-XL, it seems that hydroxyl groups located near the cross-links remain free, because of the hindrance from the cross-linking agent, and hence, serve as sites for water uptake, in addition to other accessible hydroxyl groups located on the surface or in the amorphous regions.

In summary, UICEL-XL is the first example of a cellulose II-based direct compression excipient that shows as good of binding properties as commercial cellulose I microcrystalline cellulose products. But, unlike commercial products, UICEL-XL also acts as an excellent disintegrant.

It should be appreciated that minor dosage and formulation modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

1. Cross-linked cellulose comprising:
cellulose II cross-linked with a cross-linking agent.

2. The cross-linked cellulose of claim 1 whereby the cross-linking agent is at least di-functional.

3. The cross-linked cellulose of claim 1 whereby the cross-linking agent is at least one agent selected from the group consisting of aldehydes, methyolated nitrogen compounds, dicarboxylic acids, polycarboxylic acids, halohydrins, epoxides, diepoxides, diisocyanates, dihalogen containing compounds, and ethylene epoxide.

4. The cross-linked cellulose of claim 3 whereby the cross-linking agent is a dialdehyde.

5. The cross-linked cellulose of to claim 1 that is incorporated into a tablet.

6. The cross-linked cellulose of claim 5 having a crushing strength of between about 20-55 kp.

7. The cross-linked cellulose of claim 1 having a disintegration time of less than about 25 seconds.

8. The cross-linked cellulose of claim 1 whereby the tablet has a degree of crystallinity of 75% or greater.

9. The cross-linked cellulose of claim 5 whereby the tablet has a specific surface area of 10 m²/g or greater.

10. A method of making cross-linked cellulose comprising:
combining a source of cellulose II with a cross-linking agent.

11. The method of claim 10 whereby the cross-linking agent is at least di-functional.

12. The method of claim 11 whereby the cross-linking agent is selected from the group consisting of aldehydes, methyolated nitrogen compounds, dicarboxylic acids, polycarboxylic acids, halohydrins, epoxides, diepoxides, diisocyanates, dihalogen containing compounds, and ethylene epoxide, and combinations of the same.

13. The method of claim 10 whereby the cellulose is combined with the cross-linking agent at a temperature ranging from about 60-130° C.

14. The method of claim 13 whereby the cellulose is combined with the cross-linking agent at a temperature of at least 100° C.

15. The method of claim 10 whereby the cellulose is combined with the cross-linking agent in a ratio of between about 1:0.1 to about 1:1 cellulose to cross-linking agent.

16. The method of claim 15 whereby the cellulose is combined with the cross-linking agent in a ratio of between about 1:0.3 to about 1:0.7 cellulose to cross-linking agent.

17. The method of claim 10 whereby the cellulose is allowed to react with the cross-linking agent for a time period of at least 2 hours.

18. The method of claim 17 whereby the cellulose is allowed to react with the cross-linking agent for a time period of between about 4 to about 9 hours.

19. The method of claim 18 whereby the cellulose is allowed to react with the cross-linking agent for a time period of at least 6 hours.

20. The method of claim 10 whereby the cellulose is reacted with the cross-linking agent in an acidic medium.

21. The method of claim 20 whereby the acidic medium comprises HCl.

22. The method of claim 10 whereby the cellulose is first soaked in water to form a suspension prior to reacting with the cross-linking agent.

23. The method of claim 10 whereby the cellulose is reacted with the cross-linking agent along with a coupling agent.

24. The method of claim 10 further including the step of filtering product obtained from the reaction of the cellulose with the cross-linking agent.

25. The method of claim 24 further including the step of washing the filtered product to a neutral pH.

26. The method of claim 25 further including the step of washing the product to a neutral pH with a water miscible organic solvent.

27. The method of claim 10 further including the step of drying the product.

28. The method of claim 27 further including the step of drying the product to 7% moisture by weight or less.
